(19) United States
(12) Patent Application Publication
(10) Pub. No.: US 2022/0252574 A1
(43) Pub. Date: Aug. 11, 2022

(54) IMMUNOMODULATORY COMPOSITIONS AND METHODS OF USING

(71) Applicant: University of Louisville Research Foundation, Inc., Louisville, KY (US)

(72) Inventors: Kevin Joseph Sokoloski, Crestwood, KY (US); V Douglas Landers, Louisville, KY (US)

(21) Appl. No.: 17/629,309

(22) PCT Filed: Jul. 22, 2020
(86) PCT No.: PCT/US2020/042967

§ 371 Date: Jan. 21, 2022

Related U.S. Application Data
(60) Provisional application No. 62/877,175, filed on Jul. 22, 2019.

(51) Int. Cl. G01N 33/50; C07K 14/005; A61P 37/02; C12N 5/071; C12N 15/86

(52) U.S. Cl. CPC G01N 33/5008; C07K 14/005; A61K 38/00; C12N 5/0687; C12N 15/86; A61P 37/02

(57) ABSTRACT

Immunomodulatory compositions that include at least one alphavirus capsid protein and methods of using such immunomodulatory compositions. In one aspect, methods of immunomodulating IL-1/TLR signaling in a cell are provided. Such methods typically include contacting the cell with an alphavirus capsid protein or a portion thereof, thereby immunomodulating IL-1/TLR signaling in the cell.

### CROSS REFERENCE TO RELATED APPLICATIONS

This application claims benefit under 35 U.S.C. § 119(e) to U.S. Application No. 62/877,175 filed Jul. 22, 2019.

### FEDERALLY SPONSORED RESEARCH OR DEVELOPMENT

This invention was made with government support under R01 AI153275 and T32 AI132146 awarded by the National Institute of Allergy and Infectious Diseases and under P20 GM125504 awarded by National Institute of General Medical Sciences. The government has certain rights in the invention.

### TECHNICAL FIELD

This disclosure generally relates to immunomodulatory compositions and methods of using such immunomodulatory compositions.

### BACKGROUND

The alphaviruses are positive-sense RNA viruses that are primarily spread via vector-competent mosquito species. Collectively, members of the Alphavirus genus are responsible for local, regional, and global outbreaks of clinically severe illness, often on a seasonal basis. The alphaviruses may be broadly classified via their predominant symptomology as either arthritogenic or encephalitic. The arthritogenic alphaviruses, such as Sindbis virus (SINV; the prototypic model alphavirus), Ross River virus (RRV), Semliki Forest virus (SFV), and Chikungunya virus (CHIKV), despite exhibiting low mortality, often cause febrile illness with debilitating multifocal arthritis. In some instances, the multifocal arthritis may last for several months to years past the resolution of the primary infection. In contrast to the arthritogenic alphaviruses, the encephalitic alphaviruses can exhibit significant morbidity and mortality, primarily in young children. Despite the clear impact of the alphaviruses on global human health and quality of life in developing and developed communities alike, there are no clinically proven antiviral therapeutics, or safe and effective vaccines to mitigate the public health threat of the alphaviruses.

An infectious alphavirus particle is relatively simple in design. Measuring approximately 70 nm in diameter, an alphavirus particle features an RNA cargo surrounded by two concentric icosahedral protein layers divided by a host derived lipid envelope. The viral glycoproteins, E1 and E2 (and in some instances E3), are ordered in an icosahedral array projecting from the external surface of the viral envelope. Several copies of the viral 6K and TF proteins are associated with the viral envelope. The C-terminal endodomain of the E2 protein interacts with the viral capsid protein (CP), which also forms an icosahedral structure that is symmetrically aligned with the viral glycoprotein spikes. The CP protein is the protein component of the nucleocapsid core, which also includes the viral genomic RNA. The entry pathway initiates with the viral E2 glycoprotein engaging with the host cell receptor, resulting in the endocytosis of the viral particle, and culminating in the delivery of the nucleocapsid core to the host cytoplasm. The nucleocapsid core then rapidly disassembles, releasing the viral CP protein and genomic viral RNA, the latter of which interacts with host factors to engage translational machinery to initiate the synthesis of the viral replicase complex.

The data presented in this study significantly contributes to the field by establishing the use of a robust discovery approach to identify alphaviral capsid protein: host protein interactions, and delineating a novel mechanism by which the host innate immune system is evaded during the earliest intracellular stages of the alphaviral lifecycle.

### SUMMARY

Alphaviruses are responsible for significant outbreaks of clinically severe disease. Despite their clear threat to public health, there are no approved or safe direct therapeutic interventions or vaccines to alleviate the burden of alphaviral infections.

The work described herein details an innovative interaction discovery approach focused on identifying protein:protein interactions of the alphaviral capsid protein. Importantly, these efforts led to the identification of a novel capsid-IRAK1 interaction that has profound effects on TLR- and IL1R-signaling. Thus, the capsid-IRAK1 interaction represents a novel means by which the activation of the host innate immune response is evaded and the host inflammatory response dysregulated. Ultimately, this study illuminates a previously unknown facet of the alphaviral host/pathogen interface, which is likely to have profound ramifications on alphaviral pathogenesis as well as other avenues.

In one aspect, methods of immunomodulating IL-1/TLR signaling in a cell are provided. Such methods typically include contacting the cell with an alphavirus capsid protein or a portion thereof, thereby immunomodulating IL-1/TLR signaling in the cell. The cell can be in culture or in vivo.

In another aspect, methods of reducing the innate immune response in an individual receiving a therapeutic is provided. Such methods typically include delivering an alphavirus capsid protein or a portion thereof to the individual, and delivering the therapeutic to the individual. Such methods typically reduce the innate immune response in the individual prior to the delivery of the therapeutic. The therapeutic can be delivered prior to, concurrently with, or after the alphavirus capsid protein (or the portion thereof) is delivered.

Representative alphavirus capsid proteins or portions thereof can be from an alphavirus such as, without limitation, SINV, Ross River, Chikungunya, Eastern Equine Encephalitis, and Venezuelan Equine Encephalitis. Representative therapeutics include, without limitation, polypeptides, nucleic acids, lipids, polysaccharides, carbohydrates, chemical products, plant extracts, antibodies, enzymes, growth factors, hormones, small molecules, vitamins, minerals, or any mixture or derivatives thereof.

In still another aspect, methods of screening for compounds that inhibit the infection of a host cell by an alphavirus are provided. Such methods typically include contacting an alphavirus capsid protein or a portion thereof and an IRAK1 protein or a portion thereof with a test compound. Generally, a test compound that inhibits the binding between an alphavirus capsid protein or a portion thereof and an IRAK1 protein or a portion thereof or reduces the amount of alphavirus capsid protein or a portion thereof that is bound to an IRAK1 protein or a portion thereof is indicative of a compound that inhibits the infection of a host cell by an alphavirus.

Representative alphavirus capsid proteins or portions thereof can be from an alphavirus such as SINV, Ross River, Chikungunya, Eastern Equine Encephalitis, or Venezuelan Equine Encephalitis. Representative test compounds include, without limitation, polypeptides, nucleic acids, lipids, polysaccharides, carbohydrates, chemical products, plant extracts, antibodies, enzymes, growth factors, hormones, small molecules, vitamins, minerals, or any mixture or derivatives thereof.

In some embodiments, the IRAK1 protein or the portion thereof is a human IRAK1 protein or portion thereof. The contacting can be performed in vitro or in a cell. In some embodiments, the inhibition or reduction by the test compound is at least 10%.

In yet another aspect, methods of screening for compounds that inhibit the innate immune system in an individual are provided. Such methods typically include contacting an IRAK1 protein or a portion thereof with a test compound. Generally, a compound that binds to the IRAK1 protein or the portion thereof is indicative of a compound that inhibits the innate immune system in an individual.

Such a method can further include determining the signaling capability of the IRAK1 protein or the portion thereof in the presence of the text compound. Generally, a compound that inhibits or reduces IRAK1-dependent signaling is indicative of a compound that inhibits the innate immune system in an individual.

Representative test compounds include, without limitation, polypeptides, nucleic acids, lipids, polysaccharides, carbohydrates, chemical products, plant extracts, antibodies, enzymes, growth factors, hormones, small molecules, vitamins, minerals, or any mixture or derivatives thereof. The contacting is performed in vitro or in a cell. In some embodiments, the inhibition or reduction by the test compound is at least 10%.

In another aspect, methods of screening for compounds that inhibit the infection of a host cell by an alphavirus are provided. Such methods typically include contacting an alphavirus capsid protein or a portion thereof and a protein or a portion thereof from Table 1 with a test compound. Generally, a test compound that inhibits the binding between an alphavirus capsid protein or a portion thereof and a protein or a portion thereof from Table 1 or reduces the amount of alphavirus capsid protein or a portion thereof that is bound to a protein or a portion thereof from Table 1 is indicative of a compound that inhibits the infection of a host cell by an alphavirus.

Representative alphavirus capsid proteins or portions thereof can be from an alphavirus such as, for example, SINV, Ross River, Chikungunya, Eastern Equine Encephalitis, and Venezuelan Equine Encephalitis. Representative test compounds include, without limitation, polypeptides, nucleic acids, lipids, polysaccharides, carbohydrates, chemical products, plant extracts, antibodies, enzymes, growth factors, hormones, small molecules, vitamins, minerals, or any mixture or derivatives thereof.

In still another aspect, an article of manufacture is provided that includes at least one alphavirus capsid protein or a portion thereof. Representative alphavirus capsid proteins or portions thereof can be from an alphavirus such as, for example, SINV, Ross River, Chikungunya, Eastern Equine Encephalitis, and Venezuelan Equine Encephalitis.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A-1C shows the identification of the host/pathogen interactions of the SINV capsid protein. FIG. 1A is a schematic of the BioID2 fusion proteins expressed in 293HEK cells via plasmid transfection. Individual domains are labeled above. The line in the SINV CP BioID2 construct represents a poly-glycine linker, and the green box represents a cMyc tag. FIG. 1B is a representative blot of 293HEK cell lysates after the BioID2 approach. Briefly, transfected or control transfected cells were cultured in the presence of excess biotin prior to the generation of whole cell lysates. Equal protein amounts were resolved using SDS-PAGE, and subsequently probed for protein biotinylation using streptavidin-HRP. FIG. 1C is a Venn diagram of the host proteins identified by mass spectrometry after the BioID2 approach designated the host factors as either nonspecific or specific to either BioID2 transfection/purification.

FIG. 2 shows the ontological analysis of the SINV CP:protein interactants revealed novel host/pathogen interfaces. FIG. 2A is a comparative analysis of arbitrary protein abundance of the host proteome (293HEK), and the nonspecific and CP specific interactants identified via the BioID2 approach. The lines on the graph represent the median abundance within the given data set, and the CP specific interactants are indicated next to their corresponding data point. FIG. 2B is a STRINGs interaction network map of the CP specific interactants. The color and styling of the individual nodes indicates the properties of the corresponding protein as determined by ontological categorization: round=cytoplasmic localization, square=nuclear localization; round/square=shuttling protein, or found in both compartments; red=RNA associated protein; dashed outline=membrane associated. The weight of the linear connections between the individual nodes is indicative of the relative strength/confidence of the interaction. Molecular function ontological groups, as described in the text, are highlighted in a color-coded manner.

FIG. 3 is a graph showing that the CP-IRAK1 interaction is genuine, and widely conserved across the genus Alphavirus. The interaction of the alphaviral CP proteins with the host IRAK1 protein was assessed using Nanoluc-based BiMolecular Complementation (BiMC). Briefly, 293HEK cells were co-transfected with an expression plasmid encoding the human IRAK1 protein (fused to an N-terminal Nanluc fragment), and either an expression plasmid encoding an alphavirus CP protein or the BioID2 protein (fused to the complementary C-terminal fragment of Nanoluc). Forty-eight hours post transfection, the cells were assayed using the Nano-Glo Live Cell Assay system, and the luminescence of the experimental and control conditions was detected using a plate reader. The luminescent intensity of the IRAK1-CP BiMC conditions were compared relative to those of paired control reactions lacking an interacting pair of Nanluc fusion proteins. The graphed quantitative data shown is the mean of at least 5 biological replicates, with the error bar representing the standard deviation of the means. Statistical significance relative to the control reactions, with a p-value of <0.0001=****, was determined by one-way ANOVA analysis. Below the X-axis is a phylogenetic dendrogram of alphavirus CP amino acid relatedness.

FIG. 4 demonstrates that SINV infection inhibits IRAK1-dependent signaling. FIG. 4A is a diagram of the experimental approach used to test the capacity of SINV_{P726G} to inhibit IRAK1-dependent signaling in a specific manner during infection. Comparison of the curves in each panel reveals the impact of SINV infection on (FIG. 4B) TLR4 activation by Kdo2-lipid A, (FIG. 4C) TLR7 activation by CL307, (FIG. 4D) IL1R activation by rIL-1 beta, (FIG. 4E) TLR3 activation by Poly(I:C), and (FIG. 4F) TNFR activation by rTNF alpha. In all graphs, cells mock infected prior to agonist treatment are represented by blue lines and data points, and those receiving infectious SINV_{P726G} are represented by red lines and data points. All quantitative data shown is the minimum of 6 independent biological replicates conducted over several days with at least two independent SINV preparations. Quantitative data shown is the means of the biological replicates, and the error bars represent the standard deviation of the means. The connecting line represents a nonlinear regression of the underlying data, and the shaded region indicates the 95% confidence interval of the nonlinear regression. Thus, data points where the shaded regions do not intersect are statistically significant by at least a p-value of <0.05, as determined by ANOVA analysis.

FIG. 5 demonstrates that the SINV capsid protein is sufficient for inhibition of IRAK1-dependent signaling. FIG. 5A is a diagram of the experimental approach used to test the capacity of the SINV CP protein to inhibit IRAK1-dependent signaling in a specific manner. Comparison of the curves in each panel reveals the impact of SINV CP protein expression on (FIG. 5B) TLR4 activation by Kdo2-lipid A, (FIG. 5C) TLR7 activation by CL307, (FIG. 5D) IL1R activation by rIL-1 beta, (FIG. 5E) TLR3 activation by Poly(I:C), and (FIG. 5F) TNFR activation by rTNF alpha. In all graphs, cells receiving control transfections prior to agonist treatment are represented by blue lines and data points, and those receiving the SINV CP protein expression plasmid are represented by red lines and data points. All quantitative data shown is the minimum of 6 independent biological replicates conducted over several days with at least two independent plasmid preparations. Quantitative data shown is the means of the biological replicates, and the error bars represent the standard deviation of the means. The connecting line represents a nonlinear regression of the underlying data, and the shaded region indicates the 95% confidence interval of the nonlinear regression. Thus, data points where the shaded regions do not intersect are statistically significant by at least a p-value of <0.05, as determined by ANOVA analysis.

FIG. 6 demonstrates that the SINV capsid protein delivered by incoming infectious and noninfectious particles is sufficient to inhibit IRAK1-dependent signaling. FIGS. 6A, 6C, 6E and 6G represent diagrams of the co-exposure systems used to assess the impact of the incoming SINV CP proteins derived from infectious particles. Specific differences between the experimental designs are noted in each diagram. Comparison of the curves in each panel reveals the impact of the CP-IRAK1 interaction on agonist co-exposure during (FIG. 6B) delivery of the SINV CP protein from infectious particles in the presence of the TLR7 agonist, CL307, (FIG. 6D) delivery of the SINV CP protein from infectious particles in the presence of the TLR3 agonist, Poly(I:C), (FIG. 6F) delivery of the SINV CP protein from UV inactivated particles in the presence of the TLR7 agonist, CL307, and (FIG. 6H) the effect of viral entry inhibitors on the sensing of CL307 by TLR7. In all graphs, cells receiving control transfections prior to agonist treatment are represented by blue lines and data points, and those receiving the SINV CP protein expression plasmid are represented by red lines and data points. All quantitative data shown is the minimum of 6 independent biological replicates conducted over several days with at least two independent SINV preparations. Quantitative data shown is the means of the biological replicates, and the error bars represent the standard deviation of the means. The connecting line represents a nonlinear regression of the underlying data, and the shaded region indicates the 95% confidence interval of the nonlinear regression. Thus, data points where the shaded regions do not intersect are statistically significant by at least a p-value of <0.05, as determined by ANOVA analysis.

### DETAILED DESCRIPTION

Alphaviruses are arthropod-borne RNA viruses which can cause either a mild to severe febrile arthritis which may persist for months, or encephalitis which can lead to death or lifelong cognitive impairment in the elderly and children. The non-assembly molecular role(s), functions, and protein:protein interactions of the alphavirus capsid proteins have been largely overlooked, particularly for the arthritogenic alphaviruses. Here, the BioID2 biotin ligase system was used in an innovative approach to identify host factors with which the Sindbis virus (SINV) capsid protein interacts. This approach led to the discovery of several host pathogen protein:protein interactions, including a novel validated interaction between the alphaviral capsid protein and the host IRAK1 protein, which is a critical regulator of innate immune sensing and inflammation.

The alphaviral capsid protein can be divided into two domains—a largely disordered positively charged N-terminal region, and a C-terminal protease domain. The N-terminal domain of the alphaviruses exhibits considerable sequence divergence outside of unifying characteristic of being highly polybasic. The N-terminal regions of several alphaviruses have been described in more detail, and often regions associated with nucleic acid binding, Capsid protein dimerization, and packaging specificity are noted. For several alphaviruses, most notably VEEV, distinct motifs important to the biology of the capsid protein have been identified. In contrast to the N-terminal domain, the C-terminal protease domain is largely conserved amongst the members of the genus.

Importantly, the capsid-IRAK1 interaction described herein is conserved across multiple alphavirus species including arthritogenic alphaviruses (e.g., SINV, Ross River virus, and Chikungunya virus); and encephalitic alphaviruses (e.g., Eastern Equine Encephalitis and Venezuelan Equine Encephalitis viruses). The impact of the capsid-IRAK1 interaction was evaluated using a robust set of cellular model systems, leading to the discovery that the alphaviral capsid protein specifically inhibits IRAK1-dependent signaling, representing a novel means by which the alphaviruses may evade innate immune activation and dysregulate the host inflammatory process prior to viral gene expression. Altogether, these data identify novel SINV capsid protein:protein interactions, establish the capsid-IRAK1 interaction as a common interactant amongst the alphavirus capsid proteins, and delineate the molecular consequences of the capsid-IRAK1 interaction on IRAK1-dependent signaling.

Prior to this study, the identification of alphaviral capsid protein interactions with the host were limited due to the molecular nature of the alphaviral capsid protein, which unfortunately exhibits a high degree of promiscuous binding to commercially available purification resins. The net effect is substantial precipitation of the alphaviral capsid proteins in the absence of target-specific antibodies unless highly stringent binding and wash conditions are used. The requirement of rigorous precipitation conditions necessitates the formation of cross-linked complexes prior to purification, as the wash conditions identified through the literature are likely to be incompatible with the purification of native protein:protein interaction complexes. However, the formation and purification of cross-linked complexes introduces bias into the discovery efforts, as protein:protein interactions which are comparatively rare, or fleeting in nature are likely to be underrepresented or absent in the molecular “snap shot” created by modern cross-linking approaches. Thus, efforts to define the protein:protein interactions of the alphaviral capsid proteins have been stymied and, until this report, represented a critical gap in the knowledgebase.

The adaptation of the BioID2 discovery approach to identify capsid protein interactions with host proteins using the model alphavirus, SINV, suggests that the technical limitations impeding alphaviral CP protein interaction discovery have been mitigated. The BioID2 discovery approach utilizes a promiscuous biotin ligase to covalently modify proteins which come in close proximity to the BioID2 fusion protein through protein:protein interactions. The addition of a biotin moiety directly to an interactant enables its purification, independent of the alphaviral capsid protein, using streptavidin purification resin. The affinity and avidity of the biotin-streptavidin interaction enables the use of rigorous wash conditions to purify, with high specificity, the biotinylated host proteins tagged by the CP-BioID2 fusion proteins. The BioID2 biotin ligase is also capable of tagging host protein interactants whose interactions may be exceedingly rare, or those which may be highly transient, as the biotin tag durably remains after the interaction event for subsequent purification.

As demonstrated herein, the capsid protein inhibits IRAK1-dependent signaling in a highly specific manner, such that the experimental results described herein can be applied in a number of different useful applications.

For example, the experimental results described herein allow for the ability to immunomodulate cells via the IL-1 and TLR signaling pathways. Such methods typically include contacting a cell with an alphavirus capsid protein (or a portion thereof), which, as described in this document, interacts with the host IRAK1 protein, is a critical component of TLR and IL-1 signal transduction pathways. The experimental data described herein indicates that the capsid protein is capable of reducing the dose-responsiveness of two toll-like receptors (TLRs) in infection and ectopic expression tissue culture models. The alphaviral capsid protein may serve to enable the evasion of the host innate immune response by masking the detection of PAMPs via the interruption of the IRAK1-dependent signaling cascade. Surprisingly, the experimental data described herein indicates that the capsid proteins delivered from incoming viral particles, regardless of their infectious potential, were capable of inhibiting TLR7. Therefore, the alphavirus capsid protein is capable of masking PAMP detection in permissive cells and in non-permissive cells that are exposed to a capsid protein. Thus, the interaction described herein between an alphavirus capsid protein and IRAK1 can negatively impact the detection and response to PAMP/IL-1 binding.

Significantly, the magnitude of effect is linked to the level of capsid protein present in the system, as greater effects were observed in the presence of ongoing capsid protein synthesis. Therefore, in addition to being an early mechanism by which the sensing of viral PAMPs by the IRAK1-dependent TLRs may be manipulated, ongoing capsid protein expression represents a means by which IRAK1-dependent processes can be blunted during the later stages of infection.

Virtually any cell that expresses IRAK1 and the associated receptors can be immunomodulated using the methods described herein. Simply by way of example, the immunomodulated cells can be human cells or other types of mammalian cells (e.g., companion animals (e.g., dogs, cats, etc.), farm animals (e.g., bovine, porcine, ovine, etc.) or exotic animals (e.g., large cats, animals in a zoo, etc.). The methods described herein can be applied to cells in culture (e.g., primary cell cultures derived from tissue explants or biological specimens) or in vivo. In vivo immunomodulation as described herein may be useful in treating (e.g., reversing, alleviating, or inhibiting the progress of the disorder or condition to which such term applies, or one or more symptoms of such disorders or condition) any number of disease states including, without limitation, rheumatoid arthritis, osteoarthritis, type-2 diabetes, autoimmune disorders, Familial Mediterranean Fever (FMF), and Adult and Juvenile Still Disease, Schnitzler syndrome, deficiency in IL-1 receptor antagonist (DIRA), urate crystal arthritis, postmyocardial infarction heart failure, smoldering multiple myeloma, periodic fever, aphthous stomatitis, pharyngitis, and adenitis syndrome (PFAPA).

In addition, the experimental results described herein allow for the ability to reduce the innate immune response in an individual receiving, for example, a therapeutic. Such methods typically include delivering an alphavirus capsid protein (or a portion thereof) and a therapeutic to an individual. Therapeutics are known in the art and can be, without limitation, polypeptides, nucleic acids, lipids, polysaccharides, carbohydrates, chemical products, plant extracts, antibodies, enzymes, growth factors, hormones, small molecules, vitamins, minerals, or any mixture or derivatives thereof. It would be understood that a therapeutic can be delivered concurrently with an alphavirus capsid protein (or a portion thereof), or a therapeutic can be delivered before or after, or both before and after, delivery of an alphavirus capsid protein (or a portion thereof) to an individual.

The proteins and therapeutics described herein can be formulated with a pharmaceutically acceptable carrier for delivery to an individual in an effective amount. The particular formulation and the effective amount will be dependent upon a variety of factors including route of administration, dosage and dosage interval of a compound the sex, age, and weight of the individual being treated, the severity of the affliction, and the judgment of the individual's physician. See, for example Remington: The Science and Practice of Pharmacy, University of the Sciences in Philadelphia, Ed., 21^{st} Edition, 2005, Lippincott Williams & Wilkins; and The Pharmacological Basis of Therapeutics, Goodman and Gilman, Eds., 12^{th} Ed., 2001, McGraw-Hill Co.

The overarching impact of this phenomenon is the evasion of the direct and collateral activation of an innate immune response without the need for prior intracellular viral gene expression. It is likely that this evasion mechanism is highly important to viral replication and dissemination, as alphaviruses are exceptionally sensitive to the effects of type-I IFNs. Thus, while the alphaviruses have evolved several mechanisms by which the innate immune response may be limited during intracellular replication, the fact that these evasion mechanisms require the accumulation of viral proteins via ongoing viral gene expression creates the necessity of an earlier evasion mechanism to preserve the permissibility of the host environment. The evidence presented herein suggest that the capsid protein-IRAK1 interaction represents such a mechanism.

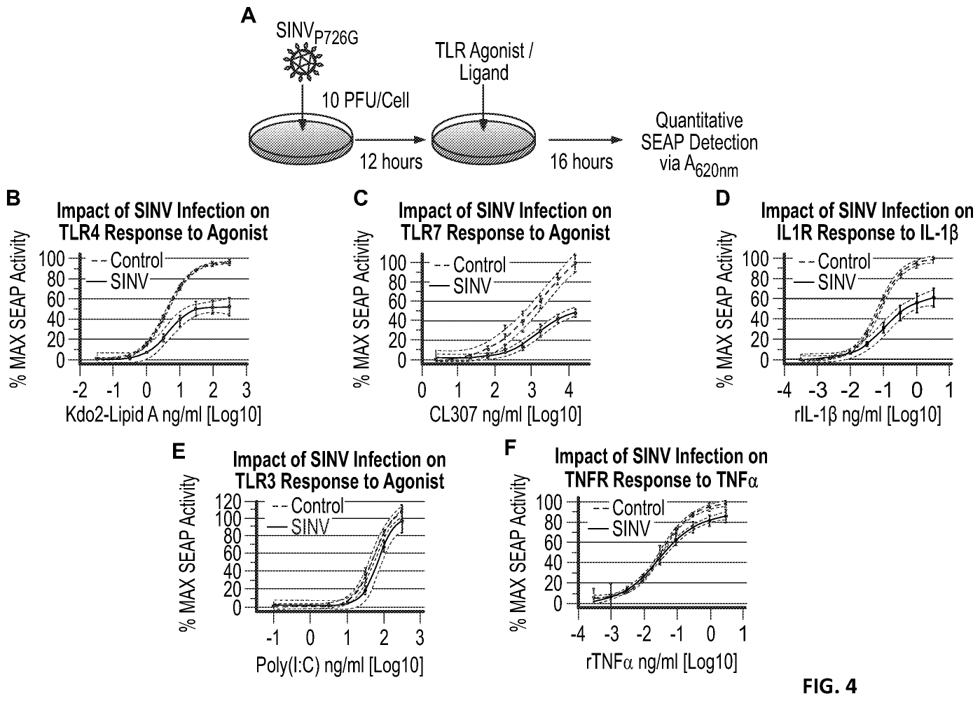

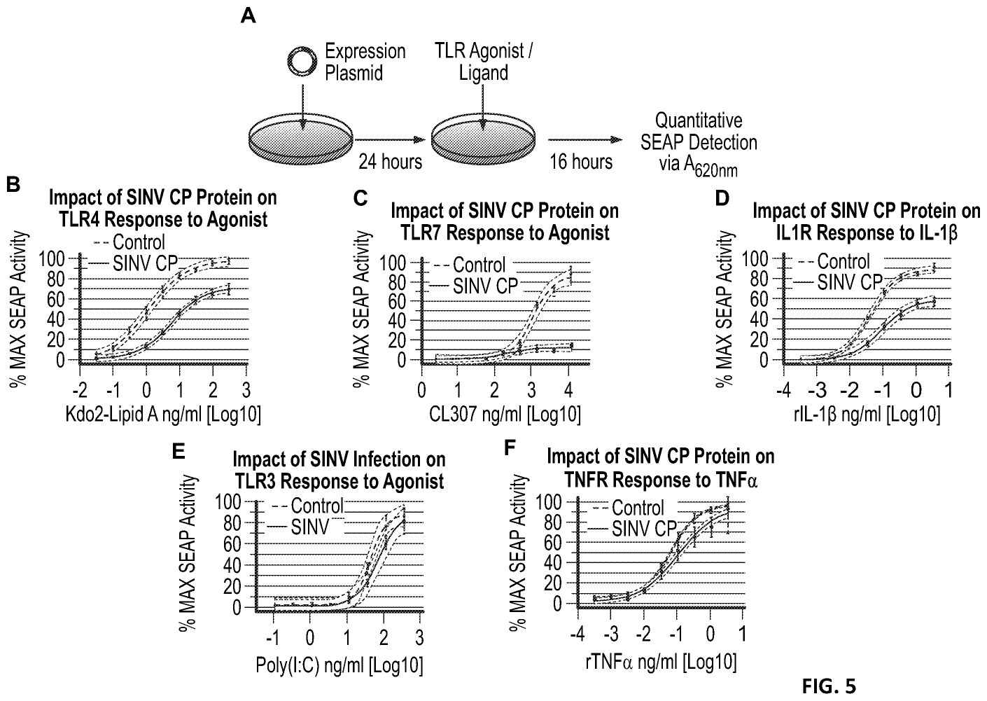

In addition to the evasion of the host innate immune response during the early stages of infection, it is hypothesized that the capsid protein-IRAK1 interaction negatively impacts IL-1 signaling during infection to dysregulate the host inflammatory response to a pathological end. As shown by the experimental data in FIGS. 4 and 5, the dose-responsiveness of the IL1R is negatively impacted by the SINV capsid protein, inferring that the capacity of the host cell to regulate the inflammatory response by sensing and responding to IL-1 is altered. As with TLR signaling, the impairment of IL1R signaling may be the product of the incoming and the nascent capsid proteins synthesized during infection. As alphaviral infection in vivo can result in the establishment of persistently infected cells, including macrophages, the capsid protein-IRAK1 interaction may represent a means by which the regulation of pro-inflammatory processes may be dysregulated, contributing to the formation of disease. It is known in the art that elevated levels of IL-1 are associated with severe alphaviral disease.

As IL-1 is a key mediator of the host inflammatory response, interfering with IL1R signaling may have profound impacts on the establishment and resolution of the inflammatory response. IL-1 has been identified as integral to the formation of arthritis and encephalitis, in both infectious and noninfectious settings. During inflammation, the activity/impact of IL-1 is controlled by balancing IL1R signaling through the expression of IL-1, IL-1 responsive genes, and IL1R-antagonists (IL1RAs). As the capsid protein-IRAK1 interaction effectively mutes IL1R signaling via an intracellular mechanism, the signals received by the binding of IL-1 to IL1R are not effectively transduced, leading to altered gene expression in infected cells.

In addition to the applications described above, the experimental results described herein also allow for compounds to be screened for their ability to inhibit the innate immune system in an individual. Such methods typically include contacting an IRAK1 protein (or a portion thereof) with a test compound. Based on the results described herein, it would be appreciated that a compound that binds to the IRAK1 protein (or a portion thereof) indicates a compound that is capable of inhibiting the innate immune system in an individual. In some instances, it may be desirable to determine the signaling capability of the IRAK1 protein (or the portion thereof) in the presence of the text compound in order to definitively identify a compound that is capable of inhibiting the innate immune system in an individual.

Further, the experimental results described herein allow for compounds to be screened for their ability to inhibit the infection of a host cell by an alphavirus. Such methods typically include combining or contacting (a) an alphavirus capsid protein (or a portion thereof); (b) an IRAK1 protein (or a portion thereof) or a protein from Table 1 (or a portion thereof); and (c) a test compound. Based on the experimental results described herein, it would be appreciated that a test compound that inhibits or reduces the binding between an alphavirus capsid protein (or a portion thereof) and an IRAK1 protein (or a portion thereof) or a protein from Table 1 (or a portion thereof) identifies those compounds that are capable of inhibiting the infection of a host cell by an alphavirus.

TABLE 1

| Gene | | |
|---|---|---|
| Designation | Function | Predictive Role in Viral Inhibition |
| LARP1 | host factor involved in the regulation | bind to mRNAs with a 5′ Terminal Oligopyrimidine |
| | of RNA stability or function | Motif (5′ Top) to prevent the association of eIf4E |
| | | with the 5′ cap structure, so the CP-LARP1 |
| | | interaction may serve to prevent LARP1 from |
| | | assembling on the viral RNA to prevent its |
| | | translation. |
| IGF2BP3 | host factor involved in the regulation | the interactions of IGF2BP3 with a given mRNA is |
| | of RNA stability or function | associated with enhanced RNA stability, therefore |
| | | this interaction may be an instance where the |
| | | recruitment of the protein to the viral RNA is |
| | | beneficial to the viral genome |
| TARDBP | host factor involved in the regulation | the association of TARDBP with an RNA has been |
| | of RNA stability or function | reported to attract elements of the cellular |
| | | deadenylase machinery, specifically Caf1, to |
| | | enhance the RNA decay in a target specific manner. |
| | | Consequently, the capsid protein-TARDBP |
| | | interaction may be another component of the |
| | | alphaviral RNA's capacity to resist deadenylation |
| | | during infection. |
| STAU1 | host factor involved in the regulation | STAU1, or Staufen1, is a component of the Staufen- |
| | of RNA stability or function | Mediated Decay (SMD) pathway, which is a highly |
| | | regulated RNA surveillance pathway, which |
| | | competes with the Nonsense Mediated Decay |
| | | (NMD) pathway. As alphaviruses have been |
| | | previously identified as prime targets for NMD, but |
| | | are apparently resistant to its effects, the interaction |
| | | of the capsid protein with STAU1 may represent a |
| | | means by which the NMD pathway is evaded during |
| | | infection. It is hypothesized that the capsid protein- |
| | | STAU1 interaction may represent a mechanism by |
| | | which the capsid protein-RNA interactions serve to |
| | | stabilize the incoming viral genomic RNAs. |
| YTHDC2 | N6-methyladenosine readers (m⁶A) | YTHDF2 contributes to the regulation of RNA |
| | | stability by recruiting the deadenylation machinery. |
| | | As such, the capsid protein may represent a means |
| | | by which the stability of the incoming viral genomic |
| | | RNA is supported. |
| YTHDF2 | N6-methyladenosine readers (m⁶A) | YTHDC2 contributes to the regulation of RNA |
| | | stability by recruiting the 5′ → 3′ exonuclease |
| | | XRN1. As such, the capsid protein may represent a |
| | | means by which the stability of the incoming viral |
| | | genomic RNA is further supported. |
| DHX30 | Zinc-finger antiviral protein (ZAP)- | DHX30 is known to associate and regulate the |
| | associated protein | activity of ZAP. Importantly, ZAP has been |
| | | previously demonstrated to restrict RNA virus |
| | | infection, including alphaviral infections. The |
| | | capsid protein-DHX30 interaction may be a means |
| | | by which the virus can evade antiviral effectors in |
| | | the inhospitable cellular environment until later |
| | | stages of infection when the host cell has been |
| | | effectively co-opted for viral replication. |

Interleukin-1 receptor-associated kinase 1 (IRAK1) proteins are known in the art and are one of two putative serine/threonine kinases that become associated with the interleukin-1 receptor (IL1R) upon stimulation. In addition, IRAK1 is partially responsible for IL1-induced upregulation of the transcription factor, NF-kappa B. The IRAK1 protein (or a portion thereof), or one of the proteins from Table 1 (or a portion thereof), can originate from virtually any organism including, without limitation, human or another type of mammal (e.g., companion animals (e.g., dogs, cats, etc.), farm animals (e.g., bovine, porcine, ovine, etc.) or exotic animals (e.g., large cats, animals in a zoo, etc.). Even the orthologous protein in an invertebrate (e.g., PELLE from Anopheles gambiae (African malaria mosquito)) interacts with the alphavirus capsid protein. Representative IRAK1 sequences can be found, for example, in GenBank Accession Nos. AAH54000.1, AAI66780.1, AAH04778.1, or NP_001035645.1.

As used herein, test compounds can include, without limitation, any organic or inorganic compound including polypeptides, nucleic acids, lipids, polysaccharides, carbohydrates, chemical products, plant extracts, antibodies, enzymes, growth factors, hormones, small molecules, vitamins, minerals, or any mixture or derivative thereof. Compounds used in the methods described herein can be natural compounds or synthetic compounds. Representative examples of compounds that can be used in the methods described herein can be found, for example, in the Sigma-Aldrich Chemical Company catalog (St. Louis, Mo.).

A person of skill in the art would appreciate that the contacting or combining any of the host or viral proteins described herein (or portions thereof) with or without a test compound can take place in vitro (e.g., in solution, in a buffer, in a cell-free system) or in a cell (e.g., in culture, in vivo), and methods of each are known in the art.

An alphavirus capsid protein (or a portion thereof) used in any of the methods described herein can be from essentially any alphavirus including but not limited to SINV, Ross River, Chikungunya, Eastern Equine Encephalitis, Venezuelan Equine Encephalitis, Eilat virus, Western Equine Encephalitis virus, Semliki Forest virus, Salmon Pancreas Disease Virus, Mayaro virus, Barmah Forest Virus. Representative sequences of alphavirus capsid proteins can be found, for example, in GenBank Accession Nos. CAA27742.1, YP_009020587.1, YP_003324595.1, YP_002802305.1, NP_740691.1, NP_818998, NP 690589, NP_740644, YP_006732328, NP_740691.1, NP_740682, NP_740639, NP_819004, NP_740673, NP_740700, and NP_818938.

As used herein, a portion of a protein typically refers to a portion of the protein that retains the ability to bind to a partner protein or retains functional activity. A skilled artisan would be able to readily determine whether a portion of a protein is suitable for use in the methods described herein.

A test compound can inhibit or reduce binding between the alphavirus capsid protein (or a portion thereof) and the IRAK1 protein or another protein shown in Table 1 (or a portion thereof) by at least 10% (e.g., at least 20%, 25%, 30%, 40%, 500%, 60%, 70%, 75%, 80%, 85%, 90%, 95%, 99% or 100%). Methods of evaluating binding are known in the art.

At least one alphavirus capsid protein (or a portion thereof) as described herein can be packaged in an article of manufacture. An article of manufacture also can include one or more therapeutics as described herein for therapeutic applications or an IRAK1 protein (or a portion thereof) as described herein and/or one or more test compounds as described herein for screening applications.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1-Tissue Culture Cells. Plasmids. Virological, and Cytological Reagents

Tissue Culture Cells

HEK293 (ATCC #CRL-1573) and BHK-21 (ATCC #CCL-10) cells were cultured in Minimal Essential Media (MEM; Cellgro), supplemented with 10% Fetal Bovine Serum (FBS; Corning), 1× Penicillin/Streptomycin (Pen/Strep; Corning), 1× Non-essential Amino Acids (NEAA; Corning), and L-glutamine (Corning). HEK293-derived reporter cells, namely HEK-Blue hTLR3, HEK-Blue hTLR4, and HEK-Blue hTLR7 (Invivogen), were cultured in Dulbecco's Modified Eagle Medium (DMEM; Corning) supplemented with 4.5 g/L glucose, 10% FBS, 1× Pen/Strep, and 1× Normocin (Invivogen). To maintain genetic homogeneity, the HEK-Blue tissue culture cells were maintained at low passage number, and supplemented with the appropriate selection antibiotics on alternating passages to maintain genomic integrity (as indicated by Invivogen's instructions per each cell line). All cell lines were cultured in humidified tissue culture incubators at 37° C. in the presence of 5% CO₂.

Plasmids

The vertebrate expression plasmids for the BioID2 screen were independently constructed, but based off those previously utilized by Kim et al. (2016, Mol. Biol. Cell., 27:1188-96). Specifically, the pBioID2-Only, and pSINVCP-BioID2 plasmids were generated via the Gibson Assembly of DNA fragments encoding a cMyc-tagged BioID2 biotin ligase and the CP protein from SINV (strain AR86) into the pCDNA3. I/Zeo(+) expression vector. To enhance the stability of the SINV CP-BioID2 fusion protein, the protease activity of the alphaviral CP proteins was eliminated by the mutation of an essential enzymatically active residue required for protease activity (Melancon et al., 1987, J. Virol., 61:1301-9).

The vertebrate expression plasmids utilized in the Nanoluc BiMolecular Complementation studies described herein were independently constructed, but based on those previously identified by Mo et al. (2017, Mol. Pharmacol., 91:339-47). Briefly, the Nanoluc protein was subdivided into two complementing fragments followed by a poly-Glycine linker. N⁶⁷, which is the N-terminal 67 amino acids of the Nanoluc protein, and C⁶⁷, which is the remaining amino acid residues, were subcloned via Gibson Assembly reactions into the vertebrate expression vector pCDNA3.1/Zeo(+). The resulting plasmids, pSplit.Nanoluc.N67 and pSplit.Nanoluc.C67, then were used in further Gibson Assembly reactions to create the plasmids used in this study. Briefly these included pSplit.Nanoluc.N67.huIRAK1, which included the full length human IRAK1 ORF, and the pSplit.Nanoluc.C67.SINV CP; RRV CP; EEE CP; VEE CP; and CHIKV CP plasmids, which contained the full length ORFs of the respective alphaviral capsid proteins. As above, to ensure the stability of the Nanoluc fragment fusion proteins, the protease activity of the alphaviral CP proteins was eliminated by the mutation of an essential enzymatically active residue required for protease activity (Melancon et al., 1987, supra). Control plasmids, including the BioID2 ORF in lieu of either the IRAK1 or the CP proteins, were generated as nonspecific controls.

To express the native SINV CP protein in a context outside of SINV infection, a vertebrate expression plasmid encoding the wild type SINV CP protein and a mCherry reporter was generated via Gibson Assembly into the pCDNA3.1/Zeo(+) vector. Specifically, the native ORFs of the SINV CP protein and E3 protein were fused to an mCherry ORF fragment to generate pEXPR.SINVCP.mCherry, which, upon transfection into a cell, directs the synthesis of a CP-E3-mCherry polyprotein which is processed into CP and E3-mCherry via the native protease activity of the SINV CP protein.

All DNA fragments for the generation of the clones above were synthesized by Genscript and assembled using the Gibson Assembly mastermix available from Synthetic Genomics, Inc. according to the manufacturer's instructions. All plasmids were verified by sequencing prior to their use in these studies. Specific plasmid information, including details regarding the restriction enzymes used for their construction; antibiotic resistance markers and bacterial growth conditions; and complete plasmid sequences, are available upon request.

All plasmids were cultured overnight in E. coli DH5α (or comparable) bacteria under antibiotic selection, and purified via miniprep or midiprep purification kits (Omega Bio-Tek). The purified plasmid DNA was phenol chloroform-extracted twice to remove any trace of endotoxin or bacterial proteins from the plasmid preparations.

Generation and Preparation of SINV

This study utilized p389_{P726G}, a Toto1101-derived SINV strain which encodes an EGFP reporter protein fused to the nsP3 gene (Frolova et al., 2006, J. Virol., 80:4122-34), and a point mutation in the nsP2 protein which abrogates the inhibition of cellular transcription (P726G) (Mayuri et al., 2008, J. Virol., 82:7284-97). The corresponding infectious clone of this SINV construct was generated via site-directed mutagenesis of the p389 infectious clone. This particular virus strain was chosen as it enables the rapid visual confirmation of viral infection, and allows for cellular transcription during infection. Infectious viral stocks were generated via the electroporation of in vitro transcribed RNA into BHK-21 cells, as previously described (LaPointe et al., 2018, MBio, 9). Briefly, ˜3×10⁶ BHK-21 cells were electroporated with 10 μg of in vitro transcribed RNA using a single pulse at 1.5 kV, 25 mA, and 200Ω. After the total infection of the monolayer (as determined by EGFP signal), the tissue culture supernatants were harvested and titered to determine the number of EGFP positive focus-forming units per ml using standard plaque assays.

For the studies utilizing non-infectious SINV particles, the aforementioned SINV reporter mutant virus was inactivated via UV irradiation (Sokoloski et al., 2013, J. Virol., 87:12216-26; Sokoloski et al., 2015, J. Virol., 89:6080-92). Briefly, 1 ml of virus stock was aliquoted into one well of a 24-well plate, placed on ice, and irradiated by exposure to 260 nm UV light in a Stratalinker for 5 minutes. The virus was promptly used and any remaining inoculum was discarded. The verification of UV inactivation was accomplished via the visualization of no EGFP signal in inoculated BHK-21 cell monolayers after 24 hours of infection.

TLR Agonists and Other Receptor Ligands

All agonists and recombinant protein ligands were diluted in pyrogen-, endotoxin-, and nuclease-free phosphate buffered saline, or distilled water, as indicated below. The reconstituted agonists/ligands were aliquoted into single use tubes and stored at −80° until use. The HEK293 TLR3 cells used in this study were stimulated with high molecular weight Poly(I:C) (Invivogen) diluted in 1×PBS. Prior to use the Poly(I:C) was heated to 75° C. and allowed to slow cool to room temperature to anneal the poly(I) and poly(C) RNA strands into dsRNA. The HEK293 TLR4 cells were stimulated with Kdo2-Lipid A (Avanti Polar Lipids) diluted in sterile nuclease free distilled water. Prior to use, the Kdo2-Lipid A was sonicated to ensure a homogenous solution prior to aliquoting and storage. The HEK293 TLR7 cells were stimulated with CL307 (Invivogen) diluted in sterile nuclease free distilled water. All of the HEK293 cells utilized in this study expressed native levels of TNFR and IL1R receptors, and were naturally responsive to stimulation with recombinant hTNFα and rIL-1β (from R&D, and Invivogen, respectively).

### Example 2—Identification of SINV CP Protein:Protein Interactions Via BioID2

To identify the Protein:Protein interactions of the SINV CP protein, a modified method derived from the previously reported BioID screens was utilized (Kim et al., 2016, Mol. Biol. Cell, 27:1188-96; Roux et al., 2018, Curr. Protoc. Protein Sci., 91:19 23 11-19 23 15; Kim et al., 2016, Methods Mol. Biol., 1411:133-46; Roux et al., 2013, Curr. Protoc. Protein Sci., 74:Unit 19 23). Per purification, approximately 2×10⁶ HEK293 cells were cultured as 80% confluent monolayers under normal conditions prior to transfection with either pBioID2-Only, or pSINVCP-BioID2. Four hours after transfection, the tissue culture medium was replaced with fresh whole growth medium supplemented with 1 μM biotin. After a 24-hour labeling incubation period, the tissue culture cells were washed with 1×PBS, and whole cell lysates were generated via the addition of Lysis Buffer (50 mM Tris, pH 7.6; 500 mM NaCl; 0.4% Sodium Dodecyl Sulfate (SDS); 1 mM DiThioThreitol (DTT); and 2.0% Triton X-100). The whole cell lysates were vortexed and frozen to ensure complete lysis, and the lysates were stored at −80° C. until ready for further use.

To verify that the BioID2 biotin ligase was working during the discovery approach, and to confirm that the biotin-labeling was specific, the whole cell lysates was assessed using SDS-PAGE and Western Blotting techniques. Briefly, equal amounts of whole cell lysates were resolved via 8% SDS-PAGE and blotted to PVDF membranes. The blotted proteins were then probed for protein biotinylation using streptavidin-HRP or anti-cMyc monoclonal antibodies to detect the individual expressed BioID2 fusion proteins.

To purify the biotinylated host proteins, the whole cell lysates were thawed on ice prior to being vigorously vortexed and clarified via centrifugation at 16,000×g for 5 minutes. The clarified whole cell lysates were transferred to a fresh microfuge tube and incubated with magnetic streptavidin beads for one hour at room temperature on a rotisserie mixer. After binding, the supernatant was removed and discarded, and the magnetic beads were washed 5 times to remove unbound proteins and nonspecific contaminants. The bound proteins were then released from the streptavidin resin via resuspension in 6× Laemmli buffer and a 15 minute incubation at 95° C.

The eluted proteins were transferred to a fresh microfuge tube and precipitated with 100% (w/v) TriChloroactic Acid (TCA) at a ratio of 1:4 (TCA:Sample). After vortexing, the samples were incubated on ice for 10 minutes to allow complete precipitation of the macromolecules in the solution. The precipitated proteins were pelleted via 5 minutes of centrifugation at 14,000×g, and the supernatant was decanted into an appropriate waste container. The protein pellet was washed three times with ice cold acetone, and the pellet was dried by incubating the microfuge tube at 95° C. for 5 minutes to drive off excess acetone.

The dried samples were resuspended in in a minimal volume of 200 mM triethylammonium bicarbonate solution (pH 8.5), and reduced via the addition of 25 mM DTT stock solution to a final concentration of 5 mM. The samples were incubated at 65° C. for 30 minutes, prior to the addition of Iodoacetamide to a final concentration of 10 mM and further incubation at room temperature in the dark for a period of 30 minutes. The alkylated samples were digested via the addition of 0.1 μg of mass spec-grade trypsin and incubation at 37° C. for 30 minutes. After the primary digestion period, a second bolus of 0.1 μg of trypsin was added and the samples were allowed to further digest overnight at 37° C. After digestion, the samples were dried in a SpeedVac and stored at −80° C.

### Example 3—the Identification of Putative SINV Capsid Interactants by Mass Spectrometry

Prior to liquid chromatography and mass spectrometry the dried samples were dissolved in 20 μl of 2% (v/v) acetonitrile and 0.1% (v/v) formic acid, and 2 μl of each sample was analyzed further. The columns used for liquid chromatography separation were an Acclaim PepMap 100 75 μm×2 cm, nanoViper (C18, 3 μm, 100 Å) trap, and an Acclaim PepMap RSLC 75 μm×50 cm, nanoViper (C18, 2 μm, 100 Å) separating column heated at 50° C. An EASY-nLC 1000 UHPLC system was used with solvents A=2% (v/v) acetonitrile/0.1% (v/v) formic acid, and B=80% (v/v) acetonitrile/0.1% (v/v) formic acid. Following injection onto the trap, the sample was separated with a 165 minute linear gradient from 0% to 55% B at 250 nL/min, followed by a 5 minute linear gradient from 55% to 95% B with a flow ramp from 250 to 300 nL/min, and lastly a 10 minute wash with 95% B at 300 nL/min. A 40 mm stainless steel emitter was coupled to the outlet of the separating column. A Nanospray Flex source was used to position the end of the emitter near the ion transfer capillary of the mass spectrometer. The ion transfer capillary temperature was set at 225° C., and the spray voltage at 1.75 kV.

An Orbitrap Elite—ETD mass spectrometer was used to collect data from the LC eluate. An Nth Order Double Play was created in Xcalibur v2.2. Scan event one obtained an FTMS MS1 scan (normal mass range; 240,000 resolution, full scan type, positive polarity, profile data type) for the range 300-2000 m/z. Scan event two obtained ITMS MS2 scans (normal mass range, rapid scan rate, centroid data type) on up to twenty peaks that had a minimum signal threshold of 5,000 counts from scan event one. Either the lock mass option was enabled (0%/o lock mass abundance) or RAW files were recalibrated offline in Xcalibur v2.2 using the 371.101236 m/z polysiloxane peak as an internal calibrant.

Proteome Discoverer v1.4.1.14 was used to analyze the data. The Sep. 27, 2018 version of the UniprotKB reviewed reference proteome canonical and isoform Homo sapiens sequences (Proteome ID UP000005640) concatenated with BioID2 and SINV-Capsid BioID2 sequences was used in the Mascot v2.5.1 and SequestHT searches. The enzyme specified was trypsin (maximum two missed cleavages with inhibition by P) with Carbamidomethyl(C) as a static modification and Oxidation(M), Biotin(K) as dynamic. Fragment tolerance was 1.0 Da (monoisotopic) and parent tolerance was 50 ppm (monoisotopic). A Target Decoy PSM Validator node was included in the Proteome Discoverer workflow.

The result files from Proteome Discoverer were loaded into Scaffold Q+S v4.4.5. Scaffold was used to calculate the false discovery rate using the Scaffold Local FDR and Protein Prophet algorithms. Peptides were accepted if the identification had probability greater than 99.9% and parent mass error within 10 ppm. Proteins were accepted if they had a probability greater than 99.9% and at least two peptides. Proteins were grouped into clusters to satisfy the parsimony principle.

The host proteins identified by the BioID2 approach were assigned as specific or non-specific on the basis of their relative detection in the BioD2-CP or BioID2 Control mass spectrometry data sets. To reduce the introduction of bias in the data sets, any relative peptide quantification data was disregarded and proteins were considered duly identified if uniquely assignable peptides were detected.

### Example 4—Bioinformatic/Ontological Analysis of Putative SINV CP Protein:Protein Interactants

To identify whether or not the host proteins identified by the BioID2 discovery approach were subject to unintentional bias on the basis of their relative protein abundance in the host proteome, the relative protein abundances of the nonspecific and SINV CP specific data sets were compared to the HEK293 proteome (Wang et al., 2015, Proteomics, 15:3163-8).

The 19 host factors identified by the SINV Capsid BioID2 discovery approach as specific to the SINV CP protein were examined using the STRING analysis (version 11.0) algorithm to detect the presence of Protein:Protein interaction networks (Szklarcyzk et al., 2017, Nucleic Acids Res., 45:D362-8). The parameters used to define the presence or absence of interaction networking included gene fusion, co-occurrence, experiments, databases, and text mining, and the confidence level was set to medium. The confidence/strength of interactions between individual host factors were scaled (arbitrarily by STRINGS version 11.0) and indicated via line weight between interconnected nodes, with higher weight indicating greater confidence.

In addition to the identification/visualization of interaction networks, the 19 putatively identified interactants were examined ontologically using DAVID to identify enriched cellular component, molecular function, and biological process ontological groups (Huang da et al., 2009, Nat. Protoc., 4:44-57; Huang da et al., 2009, Nucleic Acids Res., 37:1-13). Due to the relatively small number of host proteins in the specific group, the fold enrichment and relative statistical significances of any identified ontological groups exhibited considerable range.

The BioID2 data set is shown in Table 2.

TABLE 2

| Common | 1810 Run | 1812 Run |
|---|---|---|
| TARDBP | ZC3HAV1 | MYO1C |
| EEF1G | U2SURP | COPE |
| LTV1 | MB21D2 | IRS4 |
| DHX30 | SUGT1 | PSMD3 |
| LARS | DKC1 | XPOT |
| EIFSA | PRKCA | DNAJA2 |
| GPATCH8 | YBX1 | PHF2 |
| YTHDC2 | TJP2 | NDUFS7 |
| SMC3 | STRAP | PRKRA |
| LARP1 | DCTN1 | SURF6 |
| HSP90B1 | TMEM41B | EIF3G |
| AP2A1 | MYH9 | IPO7 |
| FBL | RPS3A | ACTL6A |
| IRAK1 | RANBP2 | ASS1 |
| IGF2BP3 | AP3D1 | ATP1A1 |
| STAU1 | EIF3D | PCCA |
| MARS | CSNK2A1 | ATP5F1B |
| YTHDF2 | NUP214 | SRPRA |
| DARS | TLN1 | EZR |
| | MDN1 | CTPS1 |
| | SMARCA5 | GART |
| | RBM25 | RPL10 |
| | ABCF1 | UQCRC1 |
| | RRBP1 | RDX |
| | H2AFY | MSH2 |
| | EIF4G1 | PSMC4 |
| | PRPF4B | MCM2 |
| | PRPF8 | HNRNPF |
| | RBBP6 | MSH6 |
| | RUVBL2 | SLC16A1 |
| | GNL2 | NPEPPS |
| | PDCD11 | GEMIN4 |
| | DNAJC9 | UBE2M |
| | PUF60 | ETF1 |
| | ANXA2 | RRP12 |
| | RBM34 | NT5DC1 |
| | RPS7 | HIST2H2BC |
| | G3BP1 | DHX57 |
| | RIF1 | ANKRD11 |
| | RBM17 | SND1 |
| | CSDE1 | EFL1 |
| | VDAC2 | DHX37 |
| | H1FX | CHD6 |
| | NT5DC2 | PDCD6IP |
| | HNRNPAB | BBX |
| | PFKL | GRWD1 |
| | HSPA9 | DDX23 |
| | NAP1L1 | USP42 |
| | RPS18 | RPF1 |
| | GPATCH4 | HEATR1 |
| | STAU2 | ARMT1 |
| | HACD3 | PPAN |
| | MRPL23 | PDLIM7 |
| | NUP188 | PAK1\|P1 |
| | SRRM1 | COPG2 |
| | COPB2 | USP16 |
| | ACSL3 | IGF2BP2 |
| | PES1 | PDLIM3 |
| | EIF2S3 | PEG10 |
| | MMTAG2 | KTN1 |
| | UBA1 | CAND1 |
| | ATP5F1C | PYCR2 |
| | HNRNPA0 | CCDC124 |
| | NOC3L | KIF1BP |
| | OLA1 | EDC3 |
| | SF3B3 | PDLIM5 |
| | URB1 | LCOR |
| | TRAP1 | BTF3L4 |
| | NACA | ZNF512 |
| | EIF4B | SLC25A3 |
| | ATP5MF | TBL3 |
| | RPS26 | AIMP1 |
| | EIF5B | AIMP2 |
| | RBBP7 | DDX10 |
| | HNRNPA1 | NAE1 |
| | ARF4 | DYNC1H1 |
| | RBM14 | MCM6 |
| | DHCR7 | TRIP12 |
| | NSFL1C | SMC1A |
| | NCLN | GANAB |
| | ILKAP | MAPRE2 |
| | HSD17B12 | SLC1A5 |
| | GATAD2A | PSMF1 |
| | | GCN1 |
| | | NUP205 |
| | | HDAC2 |
| | | UPF1 |
| | | SMARCD2 |
| | | DNAJC7 |
| | | NAP1L4 |

### Example 5—Nanoluciferase-Based BiMolecular Complementation Analysis (Nanoluc BiMC)

To validate the interaction between IRAK1 and SINV CP, an innovative BiMC approach was utilized (Mo et al., 2017, Mol. Pharacol., 91:339-47). In these experiments, HEK293 cells were seeded into flat white bottom 96 well plates at a density of 1.25×10⁴ cells per well. After an overnight incubation period, the cells were co-transfected with pSplit.Nanoluc.C67 plasmids encoding either an alphaviral CP protein or the BioID2 protein as a control, and the corresponding pSplit.Nanoluc.N67 plasmid encoding the human IRAK1 protein using Lipofectamine 3000 (Invitrogen). Specific transfection conditions for the Nanoluc BiMC assay consisted of 50 ng of each expression plasmid to achieve a total of 0.1 μg of DNA. The cells were transfected in whole growth media and incubated for a period of 48 hours under normal conditions prior to the assessment of Nanoluc complementation via the quantitative detection of Nanoluc activity via live cell NanoGlo reagents.

Briefly, to measure the levels of Nanoluc activity, the growth medium was gently removed, and replaced with 100 μl of Optimem media. Immediately after the addition of the Optimem media, NanoGlo Live Cell assay (Promega) reagents were prepared fresh as according to the manufacturer's instructions, and rapidly added to each well. The plate was briefly rocked by hand to ensure the Nanoglo reagent and cell culture media were well mixed prior to the detection of luminescence in a Synergy Hl microplate reader.

### Example 6—Quantitative Analysis of TLR, IL1R, and TNFR Signaling

Aside from the obvious differences in regards to the agonists/ligands being utilized, the overall experimental approaches used to determine the impact of the CP-IRAK1 interaction were identical irrespective of the ligand/receptor combination being tested. For all assays, the HEK293-derived reporter cell lines were cultured to ˜75% confluence in a 96-well format in whole media lacking antibiotic selection prior to being experimentally treated and assessed as follows.

To determine the impact of SINV infection on IRAK1-dependent signaling, the HEK293-derived reporter cell monolayers were either mock infected or infected with SINV_{P726G} at an MOI of 10 PFU/cell. Twelve hours post infection, the tissue culture media was removed and replaced with fresh pre-warmed whole growth medium supplemented with the indicated receptor agonists/ligands, and the tissue culture cells were returned to the incubator and incubated under normal conditions for a period of 16 hours. After the agonist/ligand activation period, the tissue culture supernatants were harvested.

To determine the impact of the SINV CP protein on IRAK1-dependent signaling, the HEK293-derived reporter cell monolayers were transfected with expression plasmids encoding either the BioID2 control plasmid or a SINV CP-E3-mCherry fusion protein capable of producing the native full length SINV CP protein after cleavage from the C-terminal E3-mCherry fusion protein. As above, 24 hours post transfection, the supernatant was removed and replaced with whole growth medium supplemented with the indicated receptor agonists/ligands, and the cells were returned to the incubator for a period of 16 hours. After the agonist/ligand activation period, the tissue culture supernatants were harvested.

To determine the impact of SINV co-exposure on IRAK1-dependent signaling, the above approach was modified. Specifically, the HEK293-derived reporter cell monolayers were either mock infected or infected with SINV_{P726G} at an MOT of 10 PFU/cell in media supplemented with the aforementioned receptor agonists/ligands for a period of 1 hour at 37° C. in a 5.0% C02 tissue culture incubator. After the co-exposure period, the treatment media was removed and the monolayers gently washed with pre-warmed whole growth medium to remove residual virus/ligand. A minimal volume of whole growth medium was added to the cell monolayers, and the cells were incubated for 16 hours prior to harvesting the tissue culture supernatants.

To define whether or not SINV replication/gene expression was required for the inhibition of IRAK1-dependent signaling, the co-exposure experiment described above was performed identically with the exception that UV-inactivated SINV particles were utilized. Similarly, to determine whether delivery of the nucleocapsid core to the host cytoplasm was required, the aforementioned co-exposure experiments utilizing infectious SINV were performed in the presence of whole growth medium supplemented with 40 μM ammonium chloride to prevent acidification of the endosome.

For all of the experimental approaches described above, the harvested tissue culture supernatants were immediately quantitatively assayed for the presence of Secreted Embryonic Alkaline Phosphatase via the use of QuantiBlue detection medium (Invivogen). Briefly, in a sterile clear-bottomed 96-well plate, 20 μl of cell-free tissue culture supernatant was added to 180 μl of QuantiBlue detection reagent and the solutions were mixed by gentle pipetting. Afterwards, the 96-well plate was incubated at 37° C. in a plate reader, and absorbance readings at 620 nm were taken every 2.5 minutes for a period of three hours, or until the A_{620 nm} curves of the highest agonist concentrations indicated saturation of the limit of detection. The A_{620 nm} readings from pre-saturation time points were comparatively assessed to determine agonist/ligand detection via the level of NFκB activation as determined by the SEAP assay colorimetric readout.

The quantitative analysis of signal transduction, as per NFκB activation, was determined by comparing the SEAP activity of the control and experimental conditions over the agonist dose range after the subtraction of un-agonist treated wells (Casella et al., 2013, PLoS One, 8:e62622; Casella et al., 2008, Cell. Mol. Life Sci., 65:3231-40). Specifically, the control agonist treatment with the highest level of relative SEAP activity within the given dose range was standardized to 100%, and all other wells were normalized accordingly to determine their relative SEAP activity to the identified maximum observed value. The quantitative data obtained from multiple biological replicates for a given dose concentration were averaged, and plotted with respect to agonist concentration. Nonlinear regression analysis of the data, via GraphPad Prism 7.0.2 using the log(agonist) vs. response—Variable slope (four parameters) nonlinear curve fit function, was used to determine the activation profiles in response to agonist treatment, and the 95% confidence intervals of the data. In addition, the agonist concentrations at which the control and experimental treatments reached 50% maximal activity (EC50_{MAX}) was determined using these nonlinear regression calculations.

### Example 7—Statistical Analyses

The quantitative data reported in this study represents the means of at least 5 biological replicates from at least two independent viral stocks, or DNA plasmid preparations, as indicated in the respective figure legend. The error bars for any given quantitative value represent the standard deviations of the means. The statistical analysis of comparative samples was accomplished using variable bootstrapping, as previously described (LaPointe et al., 2018, MBio, 9). Any p-Values for a given data set were determined via one-way ANOVA analysis, and reconfirmed using Student's t test as a post hoc analysis. Bioinformatics analyses were completed using the standard analyses of the STRING analysis (version 11.0) and DAVID gene ontology informatics suites, as described in the text.

### Example 8—the Discovery of Novel Sindbis Virus Capsid Protein:Protein Interactions

Previous work demonstrated that the SINV CP protein binds to the SINV viral genomic RNA at discrete interaction sites to accomplish non-assembly associated roles during infection (Sokoloski et al., 2017, PLoS Pathog., 13:e1006473). Further characterizations indicated that when the non-assembly SINV CP:RNA interactions were disrupted, the incoming genomic vRNAs had significantly decreased half-lives relative to wild type SINV RNAs. This led to the conclusion that the non-assembly CP:RNA interactions were involved in the regulation of viral genomic RNA stability early during infection following the disassembly of the nucleocapsid core. Nonetheless, it was postulated that the SINV CP protein was unlikely capable of directly mediating RNA stability by itself; and thus, the extent to which the SINV CP protein engaged with host factors via Protein:Protein interactions was defined.

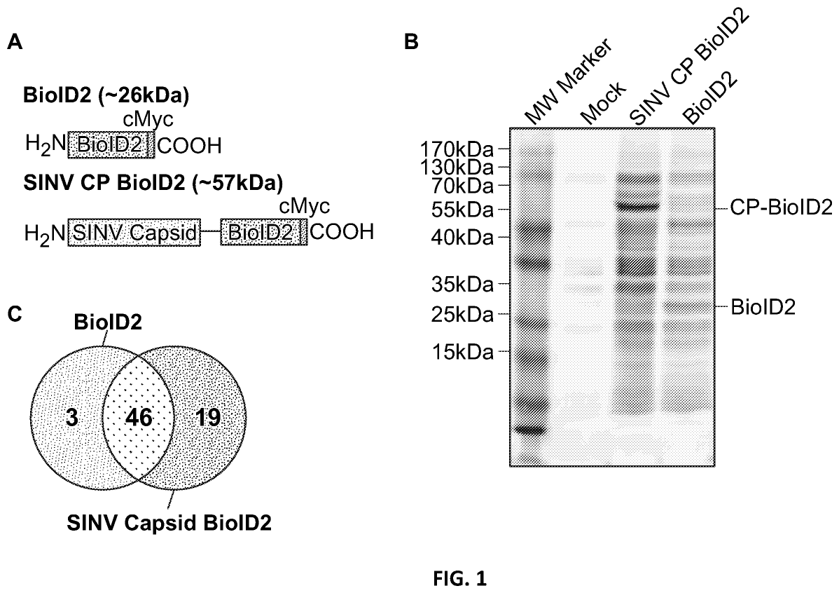

To overcome the challenges associated with working with the alphaviral CP proteins, the BioID2 discovery approach was adapted to identify SINV CP host interactions in an unbiased manner (Kim et al., 2016, Mol. Biol. Cell., 27:1188-96; Roux et al., 2018, Curr. Protoc. Protein Sci., 91:192311-5; Kim et al., 2016, Methods Mol. Biol., 1411:133-46). In this approach, the expression of BioID2 fusion proteins in the presence of excess biotin results in the labeling of protein interactants, allowing for subsequent affinity purification and identification via mass spectrometry. As depicted in FIG. 1A, the coding region of the promiscuous BioID2 biotin ligase was fused to the C-terminus of the SINV CP protein in a mammalian expression plasmid, thereby enabling the ectopic expression of a BioID2-CP fusion protein after the transfection of the BioID2-SINV CP expression plasmid in to HEK293 cells.

To test the functionality of the BioID2 biotin ligase after fusion to the SINV CP protein, whole cell lysates were generated from HEK293 cells transfected with either the BioID2-CP or BioID2-Control expression plasmids, or mock transfected, following incubation in the presence of excess biotin. Equal amounts of whole cell lysate were resolved via SDS-PAGE and transferred to PVDF prior to being probed for protein biotinylation using HRP-conjugated streptavidin (FIG. 1B). As shown by the presence of readily detectable protein species in the BioID2-CP and the BioID2-Control lanes, and the relative absence of signal in the Mock treated lane, the BioID2 biotin ligase was functional when fused to the SINV CP protein. Importantly, the overall labeling patterns of the BioID2-CP and BioID2-Control lanes exhibited unique profiles relative to one another, suggesting that the fusion of the CP protein to the BioID2 biotin ligase resulted in the specific labeling of putative CP interactants. Subsequent Western blotting with anti-Myc tag monoclonal antibodies revealed that the major protein species in either BioID2-containing transfection condition were the ectopically expressed BioID2 fusion proteins themselves, and confirmed that none of the other high molecular weight species were BioID2-CP truncation products.

To identify the host factors that engaged with the SINV CP protein during BioID2-CP expression, the aforementioned BioID2 expression plasmids were transfected into HEK293 cells and whole cell lysates were generated on a preparative scale for identification of putative interactants by mass spectrometry. As briefly described above, the biotinylated protein species from BioID2-CP and BioID2-Control whole cell extracts were purified using streptavidin resin prior to the development of trypsin digested peptide libraries for high sensitivity mass spectrometry.

In total, the two independent BioID2-CP data sets had a total of 85 and 90 unique proteins identified; whereas the BioID2-Only control had 59 and 79 unique proteins identified. Comparative analysis of the mass spectrometric data arising from two independent BioID2-CP and BioID2-Control purifications was used to identify and assign interaction specificity to putative interactants. To ensure a high degree of rigor during the discovery approach, in order to be assigned as a SINV CP protein interactant, a given host protein had to be detected in both of the SINV CP data sets and absent in either of the BioID2-Control data sets. Similarly, in order to be considered a “genuine” nonspecific BioID2 interactant, a given host protein had to be reproducibly detected in both BioID2-Control data sets. As shown in FIG. 1C, these comparative analyses revealed that a total of 68 proteins were assignable as identified interactants. Of these, 46 were identified as common between the BioID2-CP and BioID2-control, and 3 were present solely in the BioID2-Control samples, leaving 19 proteins unique to BioID2-CP (FIG. 1C).

Altogether, these data confirm that the BioID2 approach represents a means by which the host/pathogen interactions of the alphaviral CP proteins can be elucidated in a manner unrestricted by cross-linking or co-translational labeling kinetics. These efforts have led to the identification of 19 putative CP:protein interactions, which, in itself, is a significant contribution to the field, as, to date, there have been no reports of CP protein:protein interaction screens.

### Example 9—Ontological Analyses Reveal Novel Host/Pathogen Interactions

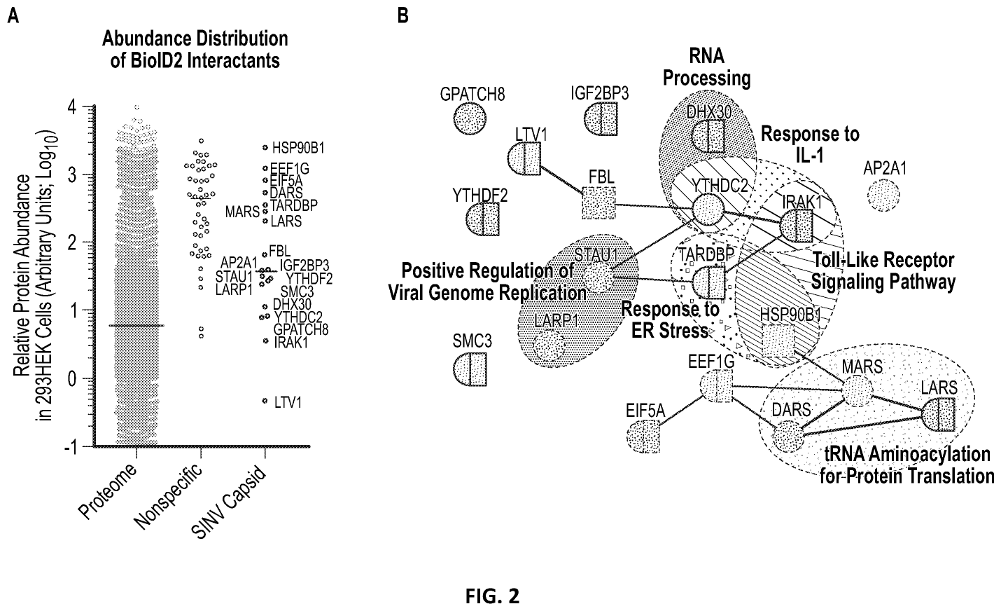

While the BioID2-CP screen led to the identification of novel SINV CP protein:protein interactions, interaction discovery screens are often subject to type-I errors. To determine the likelihood of a putative interactant being from a genuine CP:protein interaction and not a function of simple protein abundance, the data obtained from the control and SINV CP BioID2 purifications was compared with the relative protein abundances of the HEK293 proteome. This analysis, while not directly evidentiary, enables a qualitative assessment of purity by identifying whether or not a set of interactants (or an individual interactant) may be over-represented on the simple basis of protein abundance. As presented in FIG. 2A, the host factors detected and assigned as specific to the SINV CP conditions generally were of lower relative protein abundance than those identified and assigned as nonspecific interactants. Nonetheless, several of the SINV CP specific host proteins were comparable to the nonspecific interactants with respect to their arbitrary abundances in the proteome.

The 19 host factors detected during the above SINV CP BioID2 discovery approach were examined via the STRING Protein-Protein interaction network and functional enrichment analysis tool to identify common interaction networks and molecular/biological function ontologies (Szklarczyk et al., 2017, Nucleic Acids Res., 45:D362-8; Szklarczyk et al., 2015, Nucleic Acids Res., 43:D447-54). As shown in FIG. 2B, STRING analysis (Version 11.0) revealed that several of the CP protein interactants exhibited protein:protein interactions with each other independent of the CP protein, suggesting possible indirect labeling of protein complexes. Nevertheless, the group of CP interactants at large was overall devoid of extensive interrelatedness, providing an indication that the SINV CP protein interacts with host factors in a largely specific manner. Ontological analyses provided further insight into the biological functions of the SINV CP interactants. As depicted in FIG. 2B, analysis of cellular component ontology revealed that the putative interactants were associated with the Cytosol (GO:0005829), the Cytoplasm (GO:0005737), Membranes (GO:0016020), and the Nucleus (GO:0005634) to statistically significant degrees (all with p-values <0.05, with all surviving post hoc Bonferroni analyses), however the fold enrichments were ranged modestly from 2 to 4-fold. Analysis of molecular function indicated enrichment of the Poly(A) RNA Binding (GO:0044822), Protein Binding (GO:0005515), RNA Binding (GO:0003723), ATP Binding (GO:0005524), mRNA 3′-UTR Binding (GO:0003730), and Nucleic Acid Binding (GO:0003676) ontological groups. As with the analysis of cellular component ontology, each of the aforementioned groups were statistically significant by Fisher's Exact test (p-values <0.05) and survived Bonferroni post hoc analyses (with the exception of mRNA 3′-UTR Binding and Nucleic Acid Binding). Enrichment ranged from 1.8 to 10-fold amongst the FDR correction survivors.

Additionally, as highlighted in FIG. 2B, several functional clusters were identifiable amongst the putative interactants identified by the BioID2-CP screen. Notable clusters of biological function with significant enrichment (>15-fold) include the Positive Regulation of Viral Genome Replication (GO:0045070), RNA Processing (GO:0006396), Response to ER Stress (GO:0034976), tRNA Aminoacylation for Protein Translation (GO:0006418), Response to IL-1 (GO:0070555), and Toll-Like Receptor Signaling Pathway (GO:0002224) ontological groups. While the biological process GO terms listed had considerable enrichment, and initial statistical significance by Fisher's Exact test (with the exception of Response to ER Stress, and RNA Processing, where the p-values were greater than the statistical threshold of 0.05), all GO clusters succumbed to false discovery rate adjustments (likely due to the relatively few numbers of proteins in each group).

The above data indicate that the SINV CP protein is associated with a number of cytosolic RNA- and protein-binding proteins; however, these data do not indicate a singular extensive/monolithic role for the SINV CP protein in any particular cellular process outside of infection. The association of the CP protein with host factors involved in the stability of cellular RNAs is consistent with the aforementioned non-assembly roles of the SINV CP protein during infection.

The detection of the host IRAK1 protein as a putative CP:protein interaction drew significant attention due to the critical roles of the IRAK1 protein in TLR and IL1R signaling (Gottipati et al., 2008, Cell Signal., 20:269-76; Flannery et al., 2010, Biochem. Pharmacol., 80:1981-91). Previous studies have demonstrated that the host TLRs contribute to the control of alphaviral infection, as MyD88−/− mice exhibited enhanced viremia and viral dissemination relative to wild type controls (Schilte et al., 2010, J. Exp. Med., 207:429-42; Rudd et al., 2012, J. Virol., 86:9888-98). Similarly, TLR7−/− mice exhibit increased pathology and viral burdens during alphaviral infections (Neighbours et al., 2012, J. Virol., 86:10675-85). As such, given the impact of the TLRs on alphaviral infection, the CP-IRAK1 interaction was evaluated at a greater level of molecular and biological depth.

### Example 10—SINV CP-IRAK1 Interaction is Genuine, and the CP-IRAK1 Interaction is Conserved Across the Genus Alphavirus

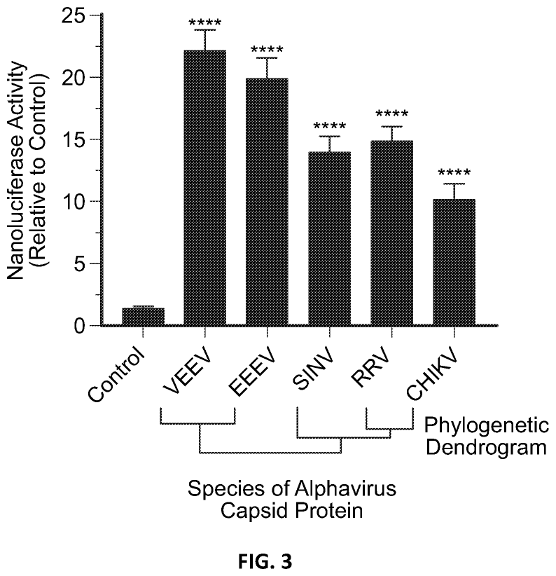

To confirm the results of the BioID experiments, a BiMolecular Complementation (BiMC) approach was used that utilized two fragments of the Nanoluc reporter (Mo et al., 2017, Mol. Pharmacol., 91:339-47). Accordingly, the N terminal BiMC fragment of Nanoluc was fused to the human IRAK1 protein, and the complementary C-terminal fragment was fused to either the SINV, Chikungunya (CHIKV), Ross River Virus (RRV), Venezuelan Equine Encephalitis Virus (VEEV), or Eastern Equine Encephalitis Virus (EEEV) capsid proteins, or the BioID2 protein as a control. To confirm and independently validate the observations of the BioID2 discovery approach, HEK293 cells were co-transfected with N-terminal Nanoluc IRAK1 plasmid and one of the above-mentioned complementary C-terminal expression plasmids. Forty-eight hours post-transfection, the cells were treated with Nano-Glo Live cell reagent and assayed for luminescence in a plate reader. As shown in FIG. 3, co-expression of the IRAK1 and SINV capsid Nanoluc BiMC proteins resulted in significantly increased Nanoluc activity relative to the control reactions, with an approximately 12-fold difference between the two experimental conditions. Similarly, co-expression of the Nanoluc-IRAK1 protein with the other aforementioned alphavirus CP proteins also significantly restored Nanoluc activity relative to the control. Specifically, the new world alphaviruses, VEEV and EEEV, demonstrated the highest BiMC activity with the human IRAK1 protein, exhibiting approximately 22-fold and 20-fold greater signal than control reactions, respectively. The CP proteins of the old world alphaviruses, RRV and CHIKV, exhibited similar BiMC profiles to SINV, with greater than 10-fold Nanoluc activity relative to control reactions.

Therefore, the IRAK1-CP interaction is conserved amongst multiple members of the alphavirus genus, and these data confirm that the CP-IRAK1 interaction identified by the BioID2 discovery approach was genuine. While the consequences of this interaction cannot be directly inferred from these data, a scenario was hypothesized in which the functionality of the IRAK1 protein was compromised by the CP-IRAK1 interaction. Given the importance of the IRAK1 protein to TLR and IL-1 signaling, the host's capacity to respond to viral infection would be significantly perturbed if the CP-IRAK1 interaction suppressed the capacity of the IRAK1 protein to function.

### Example 11—Sindbis Virus Infection Impairs IRAK1-Dependent Signaling in Tissue Culture Model Systems

Following the validation of the SINV CP-IRAK1 interaction using BiMC, it was hypothesized that the CP-IRAK1 interaction might be a means by which alphaviruses interfere with IRAK1-dependent signaling during infection to evade the induction of an antiviral innate immune response. To test this hypothesis, a series of robust/highly tractable tissue culture model systems that have been previously demonstrated to be effective in quantitatively assessing receptor activation were used (Casella et al., 2013, PLoS One, 8:e62622; Casella et al., 2008, Cell. Mol. Life Sci., 65:3231-40; Satta et al., 2011, Blood 117:5523-31; Hankins et al., 2012, Proc. Natl. Acad. Sci. USA, 109:8722-7; Salyer et al., 2016, PLoS One, 11:e0149848; Rahiman et al., 2017, Peptides, 90:48-54; Murase et al., 2018, J. Immunol., 200:2798-808; Barreto et al., 2020, Osteoarthritis Cartilage, 28:92-101; Haile et al., 2015, PLoS One, I0:e0125078; Fujita et al., 2016, Amino Acids, 48:1319-29; Grabowski et al., 2020, Biochem. Pharmacol., 177:113957; Schuster et al., 2020, Anal. Biochem., 596:113646). To this end, the impact of SINV infection on IRAK1-dependent and independent signaling events was examined using a series of 293HEK-based reporter cell lines that express Secreted Alkaline Phosphatase (SEAP) upon stimulation of TLRs 3, 4, and 7, or the IL-1 receptor via an NFκB/API-responsive promoter. Because a hallmark of alphaviral infection in highly permissive cells is the shutoff of host macromolecular synthesis, a previously established approach was employed that utilizes a SINV mutant that does not shut down host transcription, specifically the P726G point mutant of the SINV nsP2 protein to enable SEAP activation in response to agonist treatment (Mayuri et al., 2008, J. Virol., 82:7284-97).

To test the impact of SINV infection on IRAK1-dependent signaling, a battery of 293HEK-derived TLR reporter cells were infected with a Toto1101-derived SINV GFP reporter strain that included the nsP2 P726G mutation at an MOI of 10 PFU/cell. The total infection of the cell monolayer was confirmed via GFP fluorescence, and 12 hours post infection (hpi), the culture medium was replaced and the cells were treated with agonists appropriate for each target receptor over a broad dose range in half-log dilution steps. The agonist/ligand treated cells were allowed to further incubate for 16 hours post treatment (hpt) prior to the colorimetric assessment of SEAP activity in a plate reader (FIG. 4A).

SINV infection significantly impaired IRAK1-dependent signaling events, as demonstrated by decreased maximal activation and dose-responsiveness to agonist treatment for SINV infected cells relative to mock-infected controls (FIG. 4B-4D). Specifically, TLR4 reporter cells infected with SINV exhibited a ˜2-fold decrease in maximal activation relative to mock infected cells, and the amount of Kdo2-Lipid A agonist required to reach an equivalent EC50_{MAX} response of the control cells was increased by ˜12-fold (FIG. 4B). Similar results were observed for TLR7- and IL1R-signaling in TLR7 reporter cells stimulated with CL307 and rIL-1 beta, respectively. As shown in FIG. 2C, TLR7 maximal activation and dose-responsiveness were reduced by two-fold and ˜50-fold, respectively. Dose-responsiveness to rIL-1 beta treatment was similarly diminished during SINV infection, exhibiting changes akin to those observed for TLR4 treatment (FIG. 4D).

To control for the possibility that SINV infection was nonspecifically interfering with signaling/NFkB transcriptional activation during infection, the dose-responsiveness of two IRAK1 independent signaling pathways was examined after stimulation with their cognate receptor ligands. The TLR3 receptor is functionally unique amongst the TLRs in that it induces NFkB-mediated gene expression in an IRAK1 independent manner. Hence, to determine whether SINV infection non-specifically inhibited NFkB-mediated gene expression, the dose-responsiveness of TLR3 reporter cells to high molecular weight Poly(I:C) was examined. Consistent with the hypothesis that the SINV CP protein interfered with signaling in an IRAK1-dependent manner, the dose responsiveness of TLR3 was unaffected by SINV infection (FIG. 4E). Nonetheless, to further demonstrate that IRAK1 independent signaling and NFkB responsive transcription was unperturbed in each of the cellular reporter systems utilized in this study, the dose-responsiveness of TNFα Receptor (TNFR) to recombinant TNFα was examined in each of the aforementioned cell lines during SINV infection. As depicted by the data in FIG. 4F, SINV infection did not pointedly interfere with TNRR signaling, as evidenced by similar EC50_{MAX} values despite statistically significant but quantitatively modest differences at the highest concentrations of agonist. Thus, the inhibitory effects observed for IRAK1-dependent signaling events are specific and not due to simple disruption of intracellular signaling or the inhibition of NFkB/API responsive transcription/translation.

Collectively, the data presented here showed that during SINV infection, IRAK1-dependent signaling is markedly inhibited in response to TLR4, TLR7, and IL-1R stimulation, while the IRAK1-independent signaling of TLR 3 and the TNF alpha receptor was unaffected by SINV infection.

### Example 12—the Sindbis Capsid Protein is Sufficient to Inhibit IRAK1-Dependent Signaling

While the data presented in FIG. 4 was highly supportive of the initial hypothesis that the SINV CP-IRAK1 interaction represents a means by which SINV may evade the induction of a host innate immune response via the disruption of IRAK1-dependent signaling, we were unable to exclusively ascribe the disruption of IRAK1-dependent signaling to the SINV CP protein. Accordingly, to demonstrate that the SINV CP protein alone was sufficient for the disruption of IRAK1-dependent signaling, the above approach was modified to examine the impact of ectopically expressed SINV CP on IRAK1-dependent signaling.

To this end, the aforementioned 293HEK reporter cell lines were transfected with mammalian expression vectors encoding either the SINV CP protein or the BioID2 protein prior to the assessment of dose-responsiveness to agonist treatment (FIG. 5A). Consistent with the observations using SINV infection, ectopic expression of the SINV CP protein negatively impacted IRAK1-dependent signaling in a specific manner. As shown in FIG. 5B, ectopic expression of the SINV CP protein negatively impacted TLR4 signaling, as evidenced by reduced maximal activation and a >10-fold reduction in dose-responsiveness to Kdo2-Lipid A treatment. In further agreement with previous observations, TLR7 signaling was disrupted by the ectopic expression of the SINV CP protein, albeit to a strikingly more dramatic extent (FIG. 5C). Quantitative analysis of TLR7 signaling revealed that ectopic expression of the SINV CP protein effectively ablated TLR7 sensing of the CL307 agonist relative to control transfected cells. The precise mechanism underlying this phenomenon is unclear. More similar to that observed for TLR4, the capacity of the IL1R to sense rIL-1 beta was reduced by expression of the SINV CP protein, resulting in a 1.5-fold reduction in max activation and a >10-fold reduction in dose responsiveness (FIG. 5D).

To further support the conclusion that the SINV CP protein was specifically inhibiting IRAK1-dependent signaling, TLR3 and TNFR dose-responsiveness was assessed. As observed above with SINV infection, the ectopic expression of the SINV CP protein did not affect the IRAK1 independent signaling event of TLR3 (FIG. 5E). Once again, modest but statistically significant effects were observed in regards to TNFR stimulation in the presence of ectopic SINV CP protein expression, however these data may be driven by increased signal variation at the higher concentrations of rTNF alpha (FIG. 5F).

Altogether, the data presented in FIGS. 4 and 5 indicate that the SINV CP protein is capable of directly inhibiting IRAK1-dependent signaling in a highly specific manner, strongly supporting our hypothesis that the CP-IRAK1 interaction represents a means by which the host innate immune response may be evaded during infection.

### Example 13—SINV Infection Impairs IRAK1-Dependent Signaling During Viral Particles/TLR7 Agonist Co-Exposure

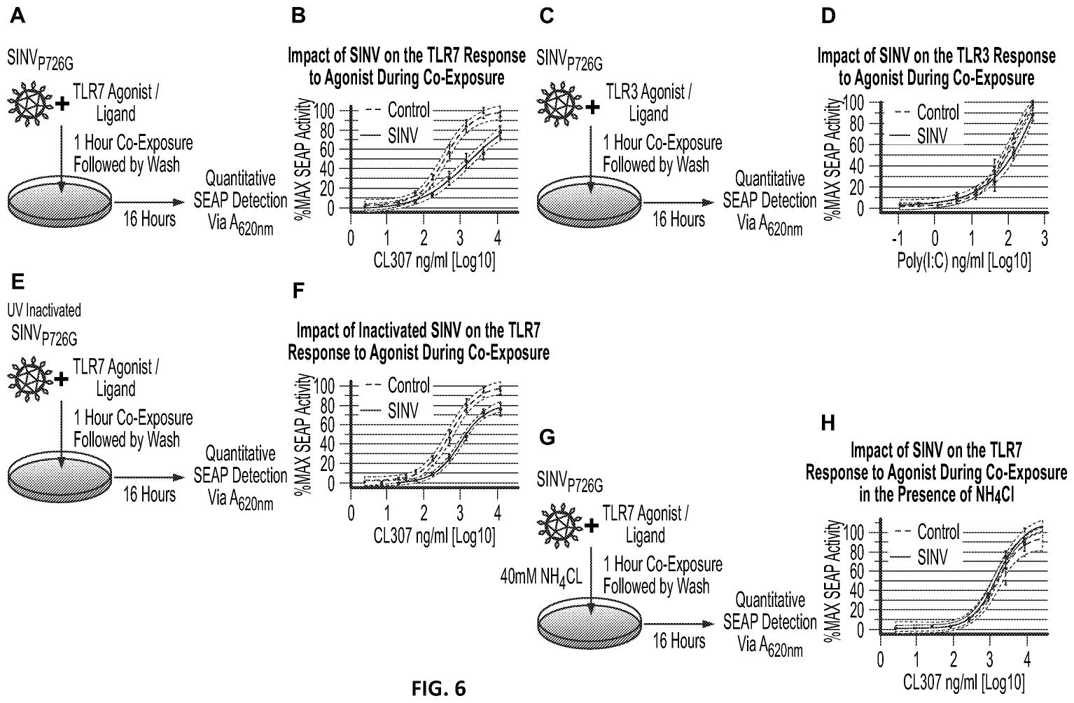

During alphaviral infection, there are two stages in which the CP protein may affect host IRAK1-dependent signaling; immediately upon entry into a new host cell, or later, during infection, when the synthesis of new CP protein has commenced. From the data obtained from the ectopic expression studies above, we can conclude that the synthesis of CP protein is capable of inhibiting IRAK1-dependent signaling events. However, the above data does not indicate whether or not IRAK1-dependent signaling is perturbed by the delivery of SINV CP protein to the cytoplasm of the target cell during viral entry. To test whether or not the SINV CP protein negatively impacted IRAK1-dependent signaling in an entry model of infection, a co-exposure system was utilized to assess the dose responsiveness of the TLR7 receptor in the presence of SINV particles (FIG. 6A). Similar to what was observed during continual SINV CP expression models, co-exposure of the TLR7 agonist with the SINV CP protein delivered by viral particles during entry reduced the extent to which IRAK1-dependent signaling was activated.

As demonstrated by the data in FIG. 6B, co-exposure of 293HEK TLR7 reporter cells with SINV particles and the TLR7 agonist CL307 elicited reduced maximal activation over the tested agonist range, and an approximate 10-fold shift in EC50_{MAX}, relative to control cells that were mock-infected during co-exposure. Nevertheless, the overall reduction of the maximal activation levels was lessened relative to systems with continual CP expression (as in FIGS. 4C and 5C). Thus, the SINV CP protein is capable of diminishing the IRAK1-dependent sensing of ssRNA PAMPs during the early stages of infection.

As before, TLR3 was utilized as a means by which the specificity of the inhibition of IRAK1-dependent signaling could be assessed during a co-exposure approach (FIG. 6C). As shown in FIG. 6D, co-exposure of Poly(I:C) and infectious SINV particles did not impact the capacity of the cells to sense and respond to TLR3 agonist. These data, in conjunction with that described above, further secure the conclusion that the SINV CP protein specifically inhibits IRAK1-dependent signaling during infection.

Careful consideration of the co-exposure approach identified the possibility that the ongoing synthesis of the SINV CP protein may be negatively impacting the capacity of the 293HEK TLR7 reporter cells to respond to agonist exposure. To control for this possibility and assess the specific impact of the incoming viral CP proteins, the co-exposure system was redesigned to utilize UV-inactivated viral particles that are incapable of initiating viral replication, and, by extension, incapable of de novo expression of the CP protein from the subgenomic RNA (FIG. 6E). In this system, any effect noted on IRAK1-dependent signaling must be due to components of the incoming viral particles. As shown in FIG. 6F, co-exposure of UV-inactivated SINV CP particles and CL307 resulted in decreased TLR7 sensing relative to mock-infected co-exposure controls. Hence, the incoming viral CP proteins delivered from non-infectious viral particles are capable of inhibiting IRAK1-dependent signaling, although not to the degree seen with longer exposure to infectious SINV.

Finally, in order to demonstrate that cytoplasmic entry of the SINV CP protein was required for the inhibition of the IRAK1-dependent TLR7 signaling process, the co-exposure system was further modified to include the co-exposure of infectious SINV particles in the presence of ammonium chloride, a lysosomotropic salt that prevents the acidification of the endosome during maturation, thereby preventing the entry of viral particles (Warner et al. 2018, Viruses, 10.PMC5850382; Helenius et al., 1982, J. Gen. Virol., 58(Pt1):47-61) (FIG. 6G). Notably, no deficiency in IRAK1-dependent signaling was observed (FIG. 6H). Therefore, these data demonstrate that endosomal acidification and the completion of the viral entry pathway, leading to release of the CP protein to the cytoplasm, is required for the inhibition of IRAK1-dependent signaling in tissue culture models of infection.

Collectively, these data provide further evidence in support of the initial hypothesis, and delineate the impacts of the CP-IRAK1 interaction on IRAK1-dependent signaling during viral entry. Moreover, these data indicate that the fusion of the viral envelope, and presumably the release of the nucleocapsid core into the cytoplasm, is required for the inhibitory effects of the incoming SINV CP protein.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A method of immunomodulating IL-1/TLR signaling in a cell, comprising:
contacting the cell with an alphavirus capsid protein or a portion thereof, thereby immunomodulating IL-1/TLR signaling in the cell.

2. The method of claim 1, wherein the cell is in culture or in vivo.

3. A method of reducing the innate immune response in an individual receiving a therapeutic, comprising:
delivering an alphavirus capsid protein or a portion thereof to the individual; and
delivering the therapeutic to the individual,
thereby reducing the innate immune response in the individual prior to the delivery of the therapeutic.

4. The method of claim 3, wherein the alphavirus capsid protein of the portion thereof is from an alphavirus selected from the group consisting of SINV, Ross River, Chikungunya, Eastern Equine Encephalitis, and Venezuelan Equine Encephalitis.

5. The method of claim 3, wherein the therapeutic is selected from the group consisting of polypeptides, nucleic acids, lipids, polysaccharides, carbohydrates, chemical products, plant extracts, antibodies, enzymes, growth factors, hormones, small molecules, vitamins, minerals, or any mixture or derivatives thereof.

6. The method of claim 3, wherein the therapeutic is delivered prior to, concurrently with, or after the alphavirus capsid protein (or the portion thereof) is delivered.

7. A method of screening for compounds that inhibit the infection of a host cell by an alphavirus, comprising:
contacting an alphavirus capsid protein or a portion thereof and an IRAK1 protein or a portion thereof with a test compound,
wherein a test compound that inhibits the binding between an alphavirus capsid protein or a portion thereof and an IRAK1 protein or a portion thereof or reduces the amount of alphavirus capsid protein or a portion thereof that is bound to an IRAK1 protein or a portion thereof is indicative of a compound that inhibits the infection of a host cell by an alphavirus.

8. The method of claim 7, wherein the test compound is selected from the group consisting of polypeptides, nucleic acids, lipids, polysaccharides, carbohydrates, chemical products, plant extracts, antibodies, enzymes, growth factors, hormones, small molecules, vitamins, minerals, or any mixture or derivatives thereof.

9. The method of claim 7, wherein the alphavirus capsid protein or the portion thereof is from an alphavirus selected from the group consisting of SINV, Ross River, Chikungunya, Eastern Equine Encephalitis, and Venezuelan Equine Encephalitis.

10. The method of claim 7, wherein the IRAK1 protein or the portion thereof is a human IRAK1 protein or portion thereof.

11. The method of claim 7, wherein the contacting is performed in vitro or in a cell.

12. The method of claim 7, wherein the inhibition or reduction by the test compound is at least 10%.

13. A method of screening for compounds that inhibit the innate immune system in an individual, comprising:
contacting an IRAK1 protein or a portion thereof with a test compound,
wherein a compound that binds to the IRAK1 protein or the portion thereof is indicative of a compound that inhibits the innate immune system in an individual.

14. The method of claim 13, further comprising determining the signaling capability of the IRAK1 protein or the portion thereof in the presence of the text compound, wherein a compound that inhibits or reduces IRAK1-dependent signaling is indicative of a compound that inhibits the innate immune system in an individual.

15. The method of claim 13, wherein the test compound is selected from the group consisting of polypeptides, nucleic acids, lipids, polysaccharides, carbohydrates, chemical products, plant extracts, antibodies, enzymes, growth factors, hormones, small molecules, vitamins, minerals, or any mixture or derivatives thereof.

16. The method of claim 13, wherein the contacting is performed in vitro or in a cell.

17. The method of claim 13, wherein the inhibition or reduction by the test compound is at least 10%.

18. A method of screening for compounds that inhibit the infection of a host cell by an alphavirus, comprising:
contacting an alphavirus capsid protein or a portion thereof and a protein or a portion thereof from Table 1 with a test compound,
wherein a test compound that inhibits the binding between an alphavirus capsid protein or a portion thereof and a protein or a portion thereof from Table 1 or reduces the amount of alphavirus capsid protein or a portion thereof that is bound to a protein or a portion thereof from Table 1 is indicative of a compound that inhibits the infection of a host cell by an alphavirus.

19. The method of claim 18, wherein the test compound is selected from the group consisting of polypeptides, nucleic acids, lipids, polysaccharides, carbohydrates, chemical products, plant extracts, antibodies, enzymes, growth factors, hormones, small molecules, vitamins, minerals, or any mixture or derivatives thereof.

20. The method of claim 18, wherein the alphavirus capsid protein or the portion thereof is from an alphavirus selected from the group consisting of SINV, Ross River, Chikungunya, Eastern Equine Encephalitis, and Venezuelan Equine Encephalitis.

21. An article of manufacture, comprising at least one alphavirus capsid protein or a portion thereof.

22. The article of manufacture of claim 21, wherein the alphavirus capsid protein or the portion thereof is from an alphavirus selected from the group consisting of SINV, Ross River, Chikungunya, Eastern Equine Encephalitis, and Venezuelan Equine Encephalitis.
